(19) United States
(12) Patent Application Publication
(10) Pub. No.: US 2010/0204629 A1
(43) Pub. Date: Aug. 12, 2010

(54) ADJUSTABLE LOWER BACK BRACE WITH POSTURE ALIGNMENT GUIDE

(76) Inventor: Greg Specht, Swansea, MA (US)

Correspondence Address: BARLOW, JOSEPHS & HOLMES, LTD., 101 DYER STREET, 5TH FLOOR, PROVIDENCE, RI, 02903, US

(21) Appl. No.: 12/540,403

(22) Filed: Aug. 13, 2009

Related U.S. Application Data
(60) Provisional application No. 61/088,464, filed on Aug. 13, 2008.

(51) Int. Cl. A61F 5/00

(52) U.S. Cl. 602/19

(57) ABSTRACT

A back brace is provided that is worn by a user for lower back support and to ease pain associated with lower back injuries and/or poor posture. The back brace of the present invention is constructed in a manner that includes a pouch formed to receive panels of semi-rigid closed cell foam that serve to adjust the thickness of the support pad and therein the support of the brace. Further, the pouch is configured to receive and retain a heat pack or a cold pack as needed by the wearer to allow the integration of heating or cooling therapy. Finally, retention members are provided on the rear of the belt and serve to receive and retain an alignment rod. The alignment rod provides a physical reference point against which the wearer can position their back when standing and when performing lifting or other posture correction exercises.

### CROSS-REFERENCE TO RELATED APPLICATIONS

This application is related to and claims priority from earlier filed U.S. Provisional Patent Application No. 61/088,464, filed Aug. 13, 2008.

### BACKGROUND OF THE INVENTION

The present invention generally relates to a back brace that is worn by a user to provide lower back support and ease pain associated with lower back injuries and/or poor posture. More specifically, the present invention relates to a lower back brace that has a back support pad of adjustable thickness, includes a pocket therein for the use of heating and/or cooling pack and includes an alignment guide feature that allows a therapist to interactively correct the wearer's posture during rehabilitation sessions and allows a user to perform independent exercise sessions to correct their posture.

When a person injures their back a brace is often employed during the rehabilitation and recovery period to provide support because the muscles in the back are employed during virtually every human movement. Typically, in order to be effective, these back braces must provide a great deal of support to the wearer. Accordingly, such back braces are either formed from a rigid plastic material or are battened with rigid strips that impart stiffness to the brace. While this construction provides tremendous support, braces formed in this manner are enormously uncomfortable for the user to wear for any extended period of time. Further, such rigid braces often hinder the wearer's ability to sit since they create so much rigidity at the wearer's mid section.

In response to the drawbacks relating to the rigid braces, elastic or soft style supports are usually preferred over rigid braces. Such soft style supports are typically used where the body part is generally healthy and the intent is to provide support and not immobilization. Further, soft supports are used to protect and promote the healing of injured members where there are no broken bones and the patient is mobile. A support may be worn, for example, before engaging in work or a sports activity that is expected to involve unusual stretching or load bearing. The elasticity of the support is important not only to provide externally applied compression, but also to maintain the support in a selected position on the body. Ideally the support is constructed so that it flexes easily and interferes as little as possible with the normal range of motion of the body part. The elasticity of the support also accommodates changes in the size of the body part produced by physical exertion.

Generally, these prior art soft braces are made of a more flexible material and include various inflatable portions or stays positioned therein that selectively increase the rigidity of the material while also providing flexibility in an attempt to increase the wearer's comfort. These devices however tend to be non-adjustable, thereby providing inappropriate support for many users. Further the stays and inflatable portions often cause pressure directly on the spine creating stress on the spinal column and nerves. In the inflatable devices, the support tends to be inadequate because under load, the device yields at the exact place where the support is required most. In addition, other devices have been proposed which allow pressure to be placed only on selected areas of the back. However, the construction of such devices is relatively complex thereby making them relatively difficult and expensive to manufacture.

Finally, while such braces provide additional support to the wearer's lower back, they are not necessarily helpful in retraining the wearer to overcome bad posture or poor lifting habits. Generally, the wearer of the brace continues the same behaviors that resulted in the back injury or weakness in the first place.

There is therefore a need for a back brace that is constructed in a manner that provides for easily adjustable support that is more uniformly distributed across the lower back region of the wearer. There is a further need for a back brace that is constructed to include a pouch that receives panels of semi-rigid closed cell foam that allow a user to adjust their spinal posture by changing the support pad thickness and rigidity. There is a further need for a back brace that includes an alignment that provides a physical reference point against which the wearer can position their back when standing and when performing lifting or other posture correction exercises.

### BRIEF SUMMARY OF THE INVENTION

In this regard, the present invention provides a back brace that is worn by a user to provide lower back support and ease pain associated with lower back injuries and/or poor posture while including a posture guide/correction feature as will be described in detail below. The back brace of the present invention is constructed in a manner that provides for easily adjustable support that is more uniformly distributed across the lower back region of the wearer.

Generally, the belt of the present invention is formed to extend around the mid section of the wearer. The belt is preferably formed from a semi-elastic material such as a neoprene that can be snuggly fit to the wearer to offer support yet includes sufficient flexibility for wearer comfort both when standing, sitting and when performing activities such as lifting, etc. The belt may be fastened using any adjustable fastening means known in the art but preferably employs hook and loop fastener strips to allow adjustment of the size and support tension. The back support also includes a user accessible pouch that is formed on the interior rear surface thereof. The pouch is formed to receive panels of semi-rigid closed cell foam that serve to adjust the thickness of the support pad and therein the support of the brace. A user can adjust their spinal posture by changing the support pad thickness by adding or removing panels of foam as desired. Further, panels having higher rigidity or more flexibility can be used to tailor the support of the brace as needed by the user.

Another feature provided by the brace of the present invention is that the pouch is configured to receive and retain a heat pack or a cold pack as needed by the wearer to allow the integration of heating or cooling therapy. In this manner the therapy pack can be inserted into the pouch thereby firmly positioning the therapy pack directly against the injured area. It should also be appreciated that the pouch may be formed to include two compartments such that the foam panels are added in the rear compartment and the therapy packs are installed in the front compartment so that the therapy pack is also supported and maintained in contact with the wearer by the rigidity of the foam panels as well.

Finally, retention members are provided on the rear of the belt and serve to receive and retain an alignment rod. During therapy sessions and while the wearer is performing self directed exercises, the alignment rod is installed into the retention members on the brace. The alignment rod provides a physical reference point against which the wearer can position their back when standing and when performing lifting or other posture correction exercises.

It is therefore an object of the present invention to provide a back brace that is constructed in a manner that provides for easily adjustable support that is more uniformly distributed across the lower back region of the wearer. It is a further object of the present invention to provide a back brace that is constructed to include a pouch that receives panels of semi-rigid closed cell foam that allow a user to adjust their spinal posture by changing the support pad thickness and rigidity. It is still a further object of the present invention to provide a back brace that includes an alignment that provides a physical reference point against which the wearer can position their back when standing and when performing lifting or other posture correction exercises.

These together with other objects of the invention, along with various features of novelty that characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a front perspective view of the back brace of the present invention;

FIG. 2 is a view of a pouch formed in the back brace of the present invention;

FIG. 3 is view of support panels being installed into the pouch in the back brace of the present invention;

FIG. 4 is a view of a therapeutic pack being installed into the poich in the back brace of the present invention;

FIG. 5 is a rear view of the back brace of the present invention;

FIG. 6 is a view of the back brace of the present invention on a user; and

FIG. 7 depicts the back brace and posture alignment guide in operation.

### DETAILED DESCRIPTION OF THE INVENTION

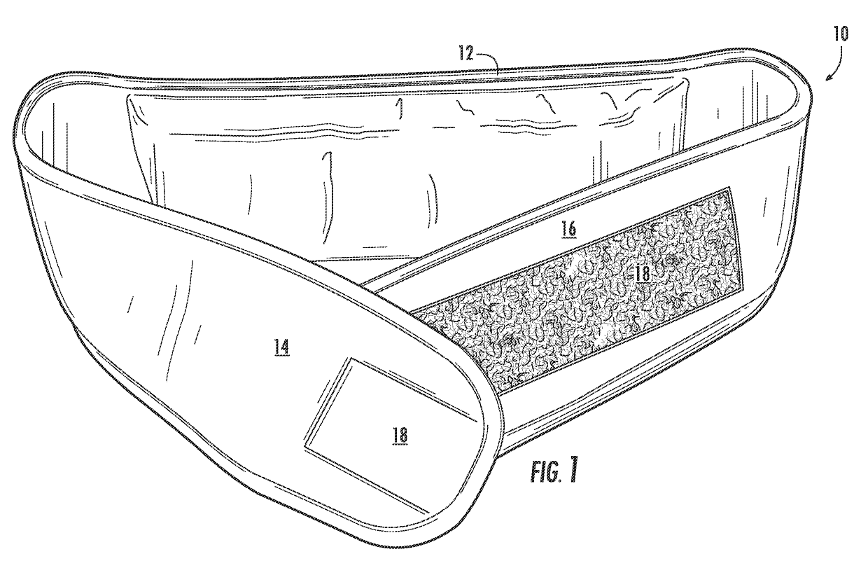

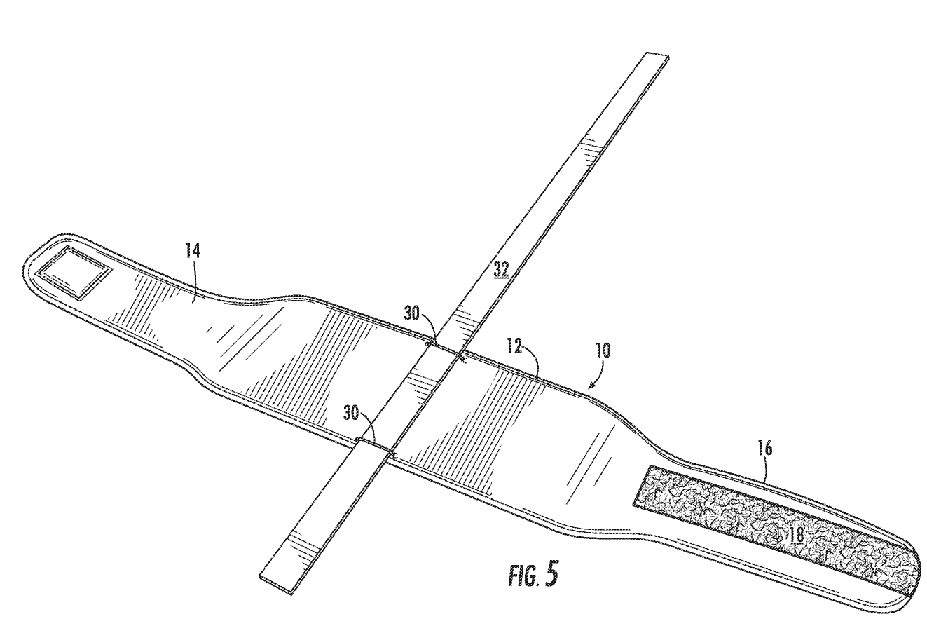

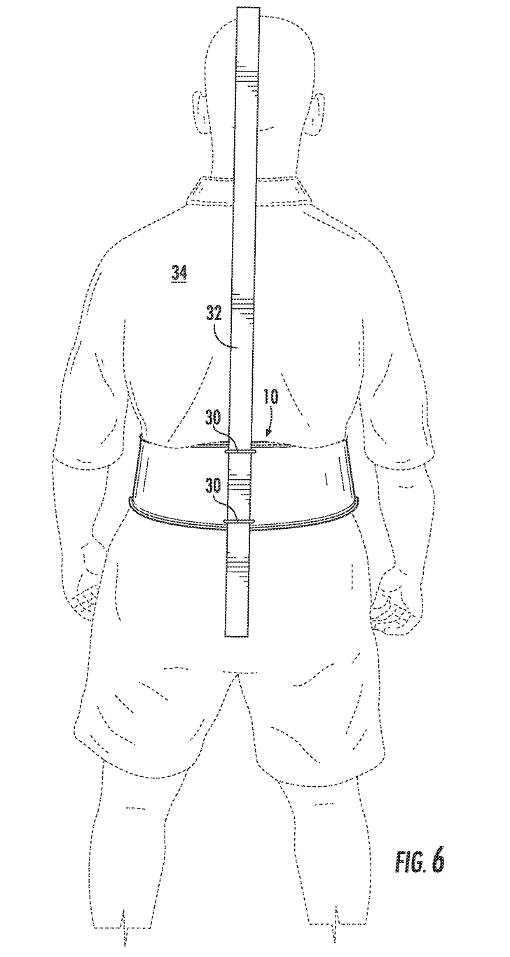

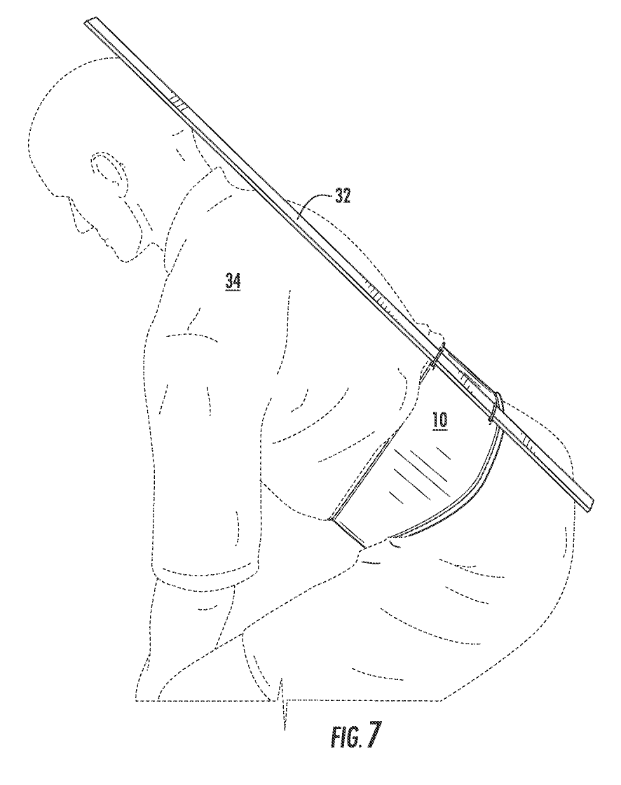

Now referring to the drawings, the back brace is shown and generally illustrated in the figures. FIGS. 1-4 generally depict the construction of the back brace itself, FIG. 5 depicts the back brace with a posture alignment guide installed thereon and FIGS. 6 and 7 depict the back brace being worn and used by a person.

As can be seen, the present invention provides for a back brace 10 that is worn by a user to provide lower back support and ease pain associated with lower back injuries and/or poor posture while including a posture guide/correction feature as will be described in detail below. Turning now to FIG. 1, the back brace 10 of the present invention is constructed in a manner that provides for easily adjustable support that is more uniformly distributed across the lower back region of the wearer. The back brace 10 of the present invention is formed to extend around the mid section of the wearer. The back brace 10 is preferably formed from a semi-elastic material such as a neoprene that can be snuggly fit to the wearer to offer support yet includes sufficient flexibility for wearer comfort both when standing, sitting and when performing activities such as lifting, etc. Further, the ability of the material to stretch allows the support tension imparted by the belt to be adjusted as the back brace 10 is placed on the user and stretched to fit. In this manner, the back brace 10 has a main body 12 that is positioned adjacent the wearer's back and includes two retention straps 14, 16 extending outwardly from the main body 12 that wrap around the wearer and attach in the front to retain the back brace 10 in position on the wearer.

As can also be seen in FIG. 1, the back brace 10 is adjustably fastened using hook and loop fastener strips 18. However, the important feature of the back brace 10 is that it can be fastened in an adjustable manner around the wearer. Accordingly, while hook and loop fasteners 18 are shown, the back brace 10 may be fastened using any adjustable fastening means known in the art to allow adjustment of the size and support tension exerted by the back brace 10.

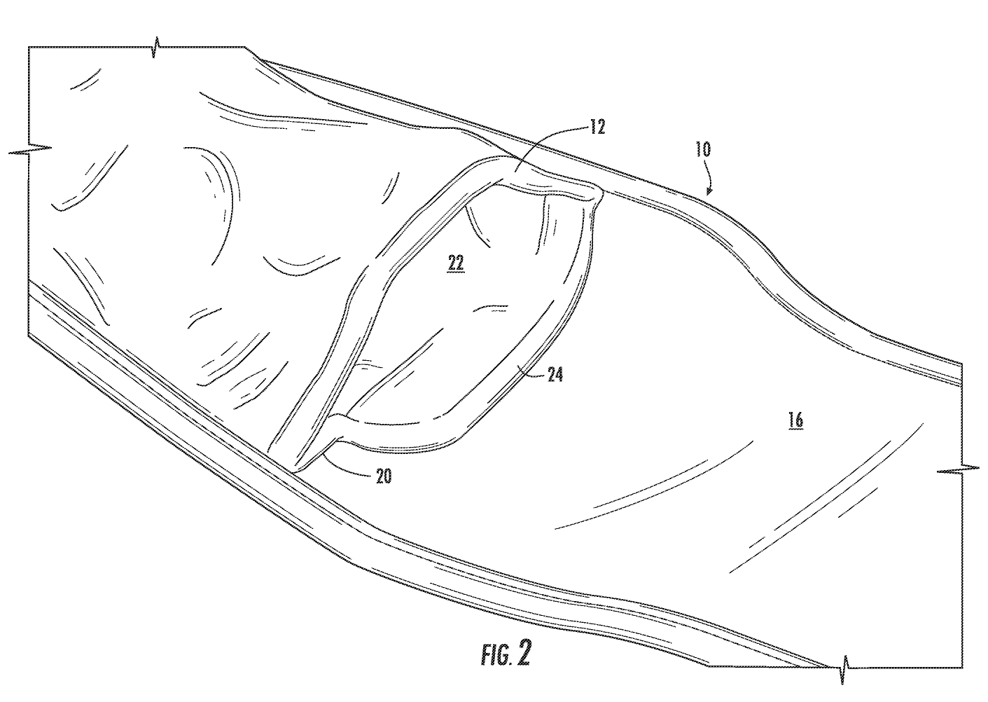

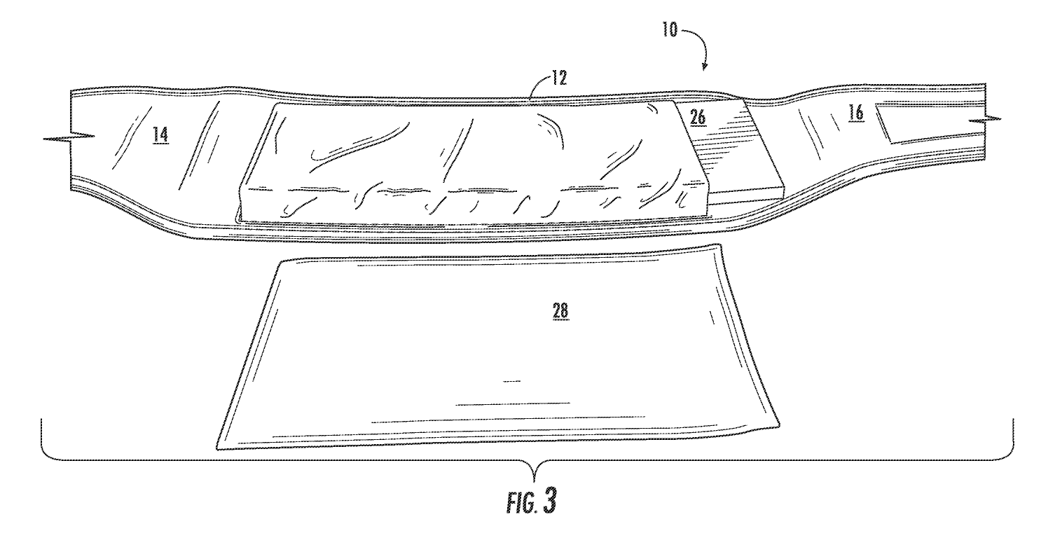

Turning now to FIGS. 2, 3 and 4, the back brace 10 of the present invention can also be seen to include at least one user accessible pouch 20 that is formed on the interior rear surface thereof. More preferably as will be described below, the back brace 10 includes at least two separate pouches 20, 22 having a dividing wall 24 positioned therebetween. As can best be seen in FIG. 3, one of the pouches 20,22 is formed to receive panels 26 of semi-rigid closed cell foam that serve to adjust the thickness of the support pad and therein the support of the back brace 10. A user can adjust their spinal posture by changing the support panel 26 thickness by adding or removing panels 26 of foam as desired. Further, panels 26 having higher rigidity or more flexibility can be used to tailor the support of the back brace 10 as needed by the user.

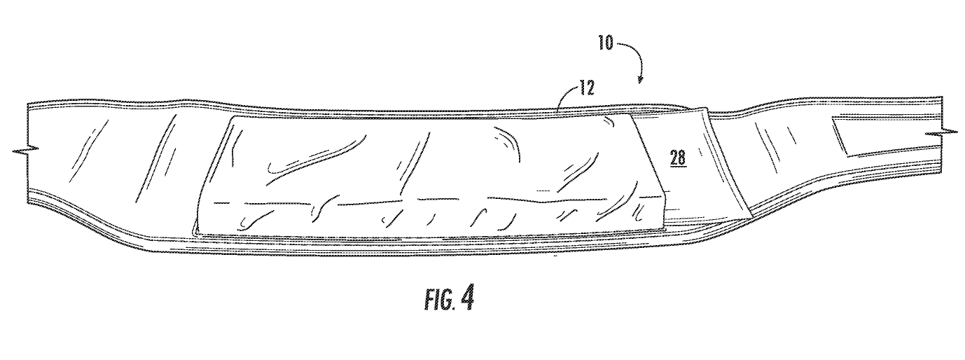

As can be seen in FIGS. 2 and 4, another feature provided by the brace of the present invention is that the secondary pouch 22 is configured to receive and retain a therapeutic pack 28 such as a heat pack or a cold pack as needed by the wearer to allow the integration of heating or cooling therapy. In this manner the therapy pack 28 can be inserted into the secondary pouch 22 thereby firmly positioning the therapy pack 28 directly against the injured area. It should also be appreciated that the pouch may be formed as one compartment or may include two compartments 20, 22 such that the foam panels 26 are added in the rear compartment 20 and the therapy packs 28 are installed in the front compartment 22. In this arrangement, the therapy pack 28 is also supported and maintained in contact with the wearer by the rigidity of the foam panels 26 as well.

Turning now to FIG. 5, the back support 10 can be seen to include retention members 30 formed on the back surface thereof. Similarly, while two loops are shown as retention members 30, one skilled in the art can appreciated that a pocket or sleeve may be formed within or on the back surface of the back brace 10 to serve the same purpose as the retention members 30. Accordingly, the particular configuration of the retention members 30 is meant to be illustrative and in no way limiting of the scope of the equivalence with respect to this feature of the invention.

Accordingly, at the center point of the rear of the back brace 10 of the present invention two retention members 30 are provided in vertical alignment with one another. The retention members 30 are positioned substantially perpendicular to the longitudinal axis of the back brace 10 in a manner that causes the retention members 30 to substantially align with the spine of the wearer when the back brace 10 is affixed to the wearer's body. The retention members 30 are shown in the drawing as elastic loops although the retention members 30 may also be snaps, straps, buckles, hook and loop fastener, hooks or any other retention member 30 suitable for the function described herein. As can be seen in FIG. 5, the retention members 30 serve to receive and retain an alignment rod 32, the function owf which will be described in detail below.

As is best illustrated in FIGS. 6 and 7, during therapy sessions and while the wearer 34 is performing self directed exercises, the alignment rod 32 is installed into the retention members 30 on the back brace 10. The alignment rod 32 provides a physical reference point against which the wearer 34 can position their back when standing and when performing lifting or other posture correction exercises. The alignment rod 32 extends above and below the back brace 10 such that the alignment rod 32 contacts the wearer 34 below the belt and above the belt to assist the wearer 34 in keeping their back properly positioned for injury prevention and or pain relief in both the front to back plane as well as in the left to right plane. In this manner, the wearer 34 is provided with immediate physical feedback concerning the use of correct posture and alignment when performing these activities.

It can therefore be seen that the present invention provides a back brace that is constructed in a manner that provides for easily adjustable support that is more uniformly distributed across the lower back region of the wearer. Further, the back brace is constructed to include a pouch that receives panels of semi-rigid closed cell foam that allow a user to adjust their spinal posture by changing the support pad thickness and rigidity and further includes an alignment guide that provides a physical reference point against which the wearer can position their back when standing and when performing lifting or other posture correction exercises. For these reasons, the instant invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A back brace, comprising:
a main body portion;
first and second retention straps extending outwardly from respective sides of said main body portion;
at least one pouch within said main body portion;
at least one support panel received and retained within said pouch; and
an alignment guide releasably attached to said main body portion.

2. The back brace of claim 1, where support imparted by said brace is adjustable by adding or removing additional support panels.

3. The back brace of claim 2, wherein said alignment guide is releasably attached to said main body by retention loops.

4. The back brace of claim 2, wherein said alignment guide is releasably attached to said main body by hook and loop fastener.

5. The back brace of claim 2, wherein said alignment guide is releasably attached to said main body by a channel formed through said main body.

6. The back brace of claim 2, wherein said alignment guide extends above and below a top and bottom edge of said main body.

7. The back brace of claim 2, wherein said support panels are formed from closed cell foam having different rigidities and thicknesses.

8. The back brace of claim 1, wherein said retention straps include fastening means that allow a tension of said back brace relative to a wearer to be adjusted.

9. The back brace of claim 8, wherein said fastening means is strips of hook and loop fastener.

10. The back brace of claim 1, wherein said alignment guide is releasably attached to said main body by retention loops.

11. The back brace of claim 1, wherein said alignment guide is releasably attached to said main body by hook and loop fastener.

12. The back brace of claim 1, wherein said alignment guide is releasably attached to said main body by a channel formed through said main body.

13. The back brace of claim 1, wherein said alignment guide extends above and below a top and bottom edge of said main body.

14. The back brace of claim 1, wherein said at least one pouch further comprises:
a forward pouch configured and arranged to receive and retain a therapy pack; and
a rearward pouch configured to receive and retain at least one support panel.

15. The back brace of claim 14, where support imparted by said brace is adjustable by adding or removing additional support panels.

16. The back brace of claim 15, wherein said support panels are formed from closed cell foam having different rigidities and thicknesses.

17. The back brace of claim 14, wherein said alignment guide extends above and below a top and bottom edge of said main body.
